SEQUENCE LISTING (6 sequences)

SEQ ID NO: 1 - RNA, 23 nt, Homo sapiens
agcaggcagaacggucuugggac

SEQ ID NO: 2 - RNA, 23 nt, Artificial Sequence
  HDAC4-MT-3′-UTR
agcaggcagaacgcagaacccuc

SEQ ID NO: 3 - DNA, 24 bp, Artificial Sequence
  miR-125a-5p miRCURYTM LNA probes
tcacaggttaaagggtctcaggga

SEQ ID NO: 4 - RNA, 23 nt, Homo sapiens
ucccugagacccuuuaaccugug

SEQ ID NO: 5 - DNA, 21 bp, Artificial Sequence
  HDAC4 shRNA-1
cgactcatcttgtagcttatt

SEQ ID NO: 6 - DNA, 21 bp, Artificial Sequence
  HDAC4 shRNA-2
gaatctgaaccactgcatttc

(19) United States
(12) Patent Application Publication
(10) Pub. No.: US 2017/0145421 A1
(43) Pub. Date: May 25, 2017

(54) MIR-125A-5P AS A BIOMARKER FOR BREAST CANCER

(71) Applicant: KAOHSIUNG MEDICAL UNIVERISTY, Kaohsiung (TW)

(72) Inventors: Eing-Mei TSAI, Kaohsiung (TW); Tsung-Hua Hsieh, Kaohsiung (TW)

(21) Appl. No.: 15/354,218

(22) Filed: Nov. 17, 2016

Related U.S. Application Data
(60) Provisional application No. 62/259,922, filed on Nov. 25, 2015.

(51) Int. Cl. C12N 15/113

(52) U.S. Cl. CPC C12N 15/1137; C12Y 305/01098; C12N 2310/141

(57) ABSTRACT

The present invention relates to a method for predicting a survival rate of a breast cancer patient, comprising: providing a biological sample of the breast cancer patient; measuring miR-125a-5p expression level in the biological sample; and comparing the miR-125a-5p expression level in the biological sample of the breast cancer patient with miR-125a-5p expression level in another biological sample of a reference breast cancer patient; wherein when the miR-125a-5p expression level in the biological sample of the breast cancer patient is lower than that of the reference breast cancer patient, the survival rate of the breast cancer patient is lower than that of the reference breast cancer patient; or when the miR-125a-5p expression level in the biological sample of the breast cancer patient is greater than that of the reference breast cancer patient, the survival rate of the breast cancer patient is greater than that of the reference breast cancer patient.

### CROSS REFERENCE TO RELATED APPLICATION

This non-provisional application claims priority from U.S. Provisional Patent Application No. 62/259,922, filed on Nov. 25, 2015, which is herein incorporated by reference in its entirety.

### FIELD OF THE INVENTION

The present invention relates to a microRNA (or miRNA) as a biomarker of breast cancer, wherein the microRNA is miR-125a-5p.

### BACKGROUND OF THE INVENTION

MicroRNAs (miRNAs) are short non-coding RNAs (19-25 nucleotides) that inhibit translation and induce mRNA degradation through binding to the 3′-untranslated region (UTR) of target mRNAs. A single miRNA can directly target many different mRNA sequences and, conversely, the same mRNA can harbor the target sites of several different miRNAs. Therefore, miRNAs and their mRNA targets constitute a regulatory network of cellular functions. In cancer cells, miRNAs are known to play critical roles in tumorigenesis by regulating cells growth, motility, angiogenesis, and apoptosis. In addition, miRNA is stably present in the serum of many cancer patients, suggesting that serum miRNA can be explored as biomarkers for cancer diagnosis and prognosis. In breast cancer, serum hsa-miR-21, miR-195, let-7a, and miR-10b have been reported as independent diagnostic and prognostic factors.

Histone deacetylases (HDACs) are the key enzymes regulating the acetylation status of both histone- and non-histone proteins. On the chromatin, HDACs play important roles in regulating chromatin stability, transcription, and replication through their activities of compacting the chromatin, and preventing the recruitment of transcription factors and RNA polymerases. In addition, by altering the acetylation status of the substrate proteins, HDACs can indirectly modulate post-translational modifications such as phosphorylation, ubiquitylation, and sumoylation, thus navigating its influence through a wide spectrum of cellular functions. Our previous study showed that HDAC6 was induced by endocrine disrupter chemicals and promoted tumorigenesis, epithelial-mesenchymal transition and angiogenesis of breast cancer. Therefore, suppressing HDACs expression is an important direction in anti-cancer drug development.

### SUMMARY OF THE INVENTION

The present invention relates to a method for predicting a survival rate of a breast cancer patient, comprising: providing a biological sample of the breast cancer patient, wherein the biological sample is a blood sample, a serum sample, or a plasma sample; measuring miR-125a-5p expression level in the biological sample; and comparing the miR-125a-5p expression level in the biological sample of the breast cancer patient with miR-125a-5p expression level in another biological sample of a reference breast cancer patient; wherein when the miR-125a-5p expression level in the biological sample of the breast cancer patient is lower than that of the reference breast cancer patient, the survival rate of the breast cancer patient is lower than that of the reference breast cancer patient; or when the miR-125a-5p expression level in the biological sample of the breast cancer patient is greater than that of the reference breast cancer patient, the survival rate of the breast cancer patient is greater than that of the reference breast cancer patient.

The present invention relates to a method for predicting a survival period of a breast cancer patient, comprising: providing a biological sample of the breast cancer patient, wherein the biological sample is a blood sample, a serum sample, or a plasma sample; measuring miR-125a-5p expression level in the biological sample; and comparing the miR-125a-5p expression level in the biological sample of the breast cancer patient with miR-125a-5p expression level in another biological sample of a reference breast cancer patient; wherein when the miR-125a-5p expression level in the biological sample of the breast cancer patient is lower than that of the reference breast cancer patient, the survival period of the breast cancer patient is lower than that of the reference breast cancer patient; or when the miR-125a-5p expression level in the biological sample of the breast cancer patient is greater than that of the reference breast cancer patient, the survival period of the breast cancer patient is greater than that of the reference breast cancer patient.

In another embodiment, when the miR-125a-5p expression level in the biological sample of the breast cancer patient is lower than that of the reference breast cancer patient, the survival period of the reference breast cancer patient is more than 5 years.

In another embodiment, when the miR-125a-5p expression level in the biological sample of the breast cancer patient is greater than that of the reference breast cancer patient, the survival period of the reference breast cancer patient is less than 1 year.

The present invention relates to an application of a miR-125a-5p for manufacturing a histone deacetylase 4 (HDAC4) inhibitor.

The present invention relates to an application of a miR-125a-5p for manufacturing a medicine for treating a breast cancer.

In another embodiment, the miR-125a-5p suppresses growth, invasion, migration, metastasis, and/or angiogenesis of the breast cancer cells.

In another embodiment, the miR-125a-5p suppresses expression of HDAC4 in the breast cancer cells.

### BRIEF DESCRIPTION OF THE DRAWINGS

The above and other features and advantages of the present invention will become more apparent by describing in detail exemplary embodiments thereof with reference to the attached drawings in which:

FIG. 1A is a statistical diagram illustrating miR-125a-5p expression level in 300 breast cancer patients' sera and tumor size thereof.

FIG. 1B is a statistical diagram illustrating overall survival rate of patients with high and low miR-125a-5p expression level of the 300 breast cancer patients.

FIG. 1C is a statistical diagram illustrating overall survival rate of patients with high and low miR-125a-5p expression level of the 177 breast cancer patients having negative lymph nodes.

FIG. 1D is a statistical diagram illustrating overall survival rate of patients with high and low miR-125a-5p expression level of the 123 breast cancer patients having negative lymph nodes.

FIG. 2A is a bar chart illustrating expression level of miR-125a-5p in different cells.

FIG. 2B is a bar chart illustrating anti-miR-125a-5p inhibits expression level of miR-125a-5p in R2N1d cells.

FIG. 2C is a bar chart illustrating miR-125a-5p inhibits R2N1d cell growth, and anti-miR-125a-5p promotes R2N1d cell growth.

FIG. 2D is a picture and a bar chart illustrating miR-125a-5p inhibits R2N1d cell migration, and anti-miR-125a-5p promotes R2N1d cell migration.

FIG. 2E is a picture and a bar chart illustrating miR-125a-5p inhibits R2N1d cell invasion, and anti-miR-125a-5p promotes R2N1d cell invasion.

FIG. 2F is an immunoblot result illustrating miR-125a-5p inhibits expression level of Ki-67 and activity of MMP2 in R2N1d cells, and anti-miR-125a-5p promotes expression level of Ki-67 and activity of MMP2 in R2N1d cells.

FIG. 3A is a sequence alignment result illustrating miR-125a-5p, the wild-type 3′-UTR of HDAC4 and the mutated 3′-UTR of HDAC4.

FIG. 3B is a bar chart illustrating miR-125a-5p suppresses luciferase reporter activity by binding to the 3′-UTR of HDAC4.

FIG. 3C is an immunoblot result illustrating miR-125a-5p inhibits HDAC4 protein level in cells, and anti-miR-125a-5p promotes HDAC4 protein levels in cells.

FIG. 3D is an in situ hybridization result illustrating miR-125a-5p expression level of breast cancer patients with different tumor grades.

FIG. 3E is an in situ hybridization result illustrating Ki-67 and HDAC4 expression level breast cancer patients with different tumor grades.

FIG. 3F is an immunoblot result illustrating HDAC4 siRNA inhibits expression level of Ki-67 and MMP2 activity.

FIG. 3G is a bar chart illustrating HDAC4 siRNA also inhibits R2N1d cell growth.

FIG. 3H is a picture and a bar chart illustrating HDAC4 siRNA also inhibits R2N1d cell migration.

FIG. 3I is a picture and a bar chart illustrating HDAC4 siRNA also inhibits R2N1d cell invasion.

FIG. 4A is a picture illustrating miR-125a-5p promotes apoptosis of R2N1d cells.

FIG. 4B is a picture and a bar chart illustrating tumor sizes of nude mice injected with R2N1d-GFP-miR-125a-5p cells or R2N1d-YFP cells; all solid arrows indicate the position for the injection of the R2N1d-YFP cells, and all dot arrows indicate the position of for the injection of the R2N1d-GFP-miR-125a-5p cells.

FIG. 4C is an immunohistochemistry result illustrating expression level of Ki-67, vascular endothelial growth factor (VEGF), and MMP2 in the tumors of nude mice injected with R2N1d-GFP-miR-125a-5p cells or R2N1d-YFP cells.

FIG. 4D is an H&E staining result illustrating the tissue section of the right lung of nude mice injected with R2N1d-GFP-miR-125a-5p cells or R2N1d-YFP cells.

FIG. 4E is a Matrigel angiogenesis assay illustrating the blood vessel and hemoglobin level of nude mice injected with R2N1d-GFP-miR-125a-5p cells or R2N1d-YFP cells.

FIG. 5A is a bar chart illustrating HDAC4 abolishes miR-125a-5p-mediated inhibition in cell growth.

FIG. 5B is a bar chart illustrating HDAC4 siRNA attenuates anti-miR-125a-5p-mediated promotion in cell growth.

FIG. 5C is a picture and a bar chart illustrating HDAC4 abolishes miR-125a-5p-mediated inhibition in cell invasion.

FIG. 5D is a picture and a bar chart illustrating HDAC4 siRNA attenuates anti-miR-125a-5p-mediated promotion in cell invasion.

FIG. 5E is a picture and a bar chart illustrating HDAC4 abolishes miR-125a-5p-mediated inhibition in cell migration.

FIG. 5F is a picture and a bar chart illustrating HDAC4 siRNA attenuates anti-miR-125a-5p-mediated promotion in cell migration.

FIG. 5G is a picture and a bar chart illustrating HDAC4 abolishes miR-125a-5p-mediated inhibition in tumor growth.

FIG. 5H is a picture and a bar chart illustrating HDAC4 siRNA attenuates anti-miR-125a-5p-mediated promotion in tumor growth.

### DETAILED DESCRIPTION OF THE INVENTION

Embodiments of the present invention are described in the following examples that are intended for illustration only. One skilled in the art would appreciate that other modifications and variations are possible without departing from the scope of the invention. Various embodiments of the invention are described in detail in the following sections, referring to the drawings, in which like numbers indicate like components throughout the views.

### Materials and Methods

### Cell Lines and Clinical Specimens

The breast cancer cell lines H184B5F/M10, MDA-MB-435, MDA-MB-231, MCF-7, and MCF-7/Her18 were purchased from American Type Culture Collection (ATCC). The cancer stem cell lines R2d and R2N1d were a kind gift from Prof. C.-C. Chang (Michigan State University, East Lansing, Mich.). Human breast cancer specimens were collected from the Kaohsiung Medical University Hospital.

### Luciferase Assay

HEK-293T cells were co-transfected with PGL3-control-3′-UTR (Promega), PGL3-HDAC4-WT-3′-UTR (SEQ ID NO:1), or PGL3-HDAC4-MT-3′-UTR (SEQ ID NO:2), and the indicated amounts of miR-125a-5p using TurboFect Transfection Reagent (Fermentas). Cells were cultured for 24 hr after transfection, and activity was measured with the Dual-Glo Luciferase Assay (Promega).

### In Vivo Experiments

Female mice (BALB/cAnN. Cg-Foxnlnu/Crl-Narl, 4 to 6 weeks old) were obtained from the National Laboratory Animal Center (Taipei, Taiwan). R2N1d cells were infected with viruses carrying pLKO.1-YFP (National RNAi Core Facility, Academia Sinica, Taipei, Taiwan) or pLKO.1-GFP-miR-125a-5p plasmids. For the xenograft model, cells stably expressing YFP or GFP-miR-125a-5p were injected subcutaneously into the flanks of nude mice, and the fluorescent density was measured 7, 14, 21, and 28 d after injection using an Ultra Sensitive Molecular Imaging System (Berthold Technologies). For the metastasis model, R2N1d-YFP or R2N1d-GFP-miR-125a-5p cells were mixed with Matrigel (BD Biosciences) and injected into the left lateral thorax of nude mice as described. The extra- and intra-thoracic lymph nodes in the right lung were quantified with a dissecting microscope and pathologically confirmed by H&E staining. For the matrigel plug angiogenesis model, the cells were resuspended and mixed with Matrigel (1:1) and then injected into the flanks of nude mice as described. Fifteen days after implantation, blood vessel formation was determined with H&E staining, and hemoglobin values were analyzed using Drabkin's reagent kit (Sigma).

### MiRNA Isolation and Quantitative Real-Time PCR

Total RNA was extracted from serum using the MasterPure Complete DNA & RNA Purification kit (EPICENTRE Biotechnologies). miRNA was amplified using the corresponding reverse transcription primer and the TaqMan MicroRNA Reverse Transcription kit (Applied Biosystems). miR-16 was used for normalization of miRNA amounts in serum, and the 2^{̂-Δct} method was used to determine the relative expression.

### In Situ Hybridization and Immunohistochemistry

For in situ hybridization, miR-125a-5p in tissues sections was detected using a 5′-digoxygenin-labeled miR-125a-5p miRCURY™ LNA detection probe (Exiqon) and an IsHyb In Situ Hybridization kit (BioChain). The probe sequence was SEQ ID NO:3, 5′-TCACAGGTTAAAGGGTCTCAGGGA-3′. For immunohistochemistry, 5-μm thick sections were deparaffinized with xylene and dehydrated using ethanol. Immunohistochemistry staining was performed with a Dako LSAB kit (Dako). The nuclei were counterstained with hematoxylin. The following antibodies were used for immunohistochemistry: HDAC Family Antibody Set (Biovision), anti-Ki-67 (Sigma), anti-VEGF (Santa Cruz Biotech), anti-MMP2 (Cell Signaling).

### Immunoblot Analysis

Cell lysates were prepared with the M-PER mammalian protein extraction reagent (Thermo Scientific) and stored at −20° C. until use. For immunoblot analysis, cell lysates were resolved on SDS/PAGE gels and blotted onto polyvinylidene difluoride membranes (Millipore). Membranes were probed with antibodies at 4° C. for 24 hr and developed with the ECL plus Western Blotting kit (Millipore). The following antibodies were used for immunoblotting: HDAC Family Antibody Set (Biovision), anti-Ki-67 (Sigma), anti-MMP2 (Cell Signaling).

Transfection and Plasmids, siRNA, and shRNA

Cells were seeded into a 6-well plate, incubated for 24 hr, and then transfected with plasmid or RNA using TurboFect Transfection Reagent (Fermentas). The following plasmid and RNAs were used:

pLKO. TRC-miR-125a-5p
(5′-UCCCUGAGACCCUUUAACCUGUG-3′; SEQ ID NO: 4),
pLKo. 1-HDAC4 shRNA-1
(5′-CGACTCATCTTGTAGCTTATT-3′; SEQ ID NO: 5),
pLKo. 1-HDAC4 shRNA-2
(5′-GAATCTGAACCACTGCATTTC-3′; SEQ ID NO: 6).

### Cell Growth, Invasion, and Wound-Healing Assays

Cell growth was assessed using the 3′-(1-(phenylaminocarbony)-3,4-tetrazolium)-bis-(4-methoxy-6-nitro)-benzene sulfonic acid hydrate (XTT) solution (Sigma), and absorbance at 490 nm was measured in an ELISA reader (Multiskan EX). For the wound healing assay, the cells were cultured for 24 h (90% confluency) and scratched with a micropipette tip in a six-well plate. 24 h later, the wound width was captured by light microscope (Olympus) and wound closure was measured at three defined positions along the scratch. The invasiveness of cells was evaluated by a Cell Invasion Assay kit (Chemicon). Briefly, the invading cells on the lower surface of the membrane were stained with crystal violet (Sigma) and photographs were captured by a microscope.

### Immunofluorescence and Apoptosis Assay

The YFP- or GFP-miR-125a-5p-expressing cells and tissues sections were fixed for 20 min in 4% paraformaldehyde. Cells were observed with an IX-71 microscope and analyzed with DP2-BSW software (Olympus). For apoptosis assay, the cells were analyzed with annexin V-FITC apoptosis kit (BD Biosciences Pharmingen). The cells were collected and stained with propidium iodide (PI) and Annexin V. After 30 min, the samples were analyzed by flow cytometry.

### Example 1

### Association of Circulation miR-125a-5p with Clinicopathological Characteristics and Prognosis in Human Breast Cancer Patients

To investigate whether miRNAs are associated with survival in patients with breast cancer, we profiled miRNA expression in serum samples from five breast cancer patients who survived for less than 1 year after diagnosis (short-survival group) and five breast cancer patients who survived for more than 5 years after diagnosis (long-survival group) using an miRNA microarray (System Biosciences). All patients had tumors positive for estrogen receptor, progesterone receptor, and HER2/ErbB2. The results showed that miR-125a-5p expression was highly different in these two groups and showed relatively low expression levels in short-term survivors (Table 1).

TABLE 1

| | Short | | | | |
|---|---|---|---|---|---|
| | Survival Group | | Long Survival Group | | |
| miRNA | Normalized | SD | Normalized | SD | Fold |
| hsa-miR-125a-5p | 0.05 | 0.08 | 1.95 | 0.02 | 0.03 |
| hsa-miR-206 | 1.64 | 0.06 | 0.07 | 0.03 | 24.37 |
| hsa-miR-146b-3p | 0.2 | 0.13 | 3.93 | 0.06 | 0.05 |
| hsa-miR-518a-3p | 0.44 | 0.07 | 0.03 | 0.02 | 16.97 |
| hsa-miR-193a-5p | 1.91 | 0.05 | 0.13 | 0.06 | 14.88 |
| hsa-miR-155 | 3.73 | 0.04 | 0.38 | 0.07 | 9.85 |
| hsa-miR-181c | 1.2 | 0.08 | 10.31 | 0.09 | 0.12 |
| hsa-miR-520c-3p | 0.07 | 0.05 | 0.63 | 0.07 | 0.12 |
| hsa-miR-30a | 4.96 | 3.54 | 0.64 | 0.15 | 7.71 |
| hsa-miR-181b | 0.08 | 0.09 | 0.63 | 0.04 | 0.13 |
| hsa-miR-503 | 24.03 | 0.07 | 3.15 | 0.06 | 7.63 |
| hsa-let-7b | 0.02 | 0.05 | 0.12 | 0.02 | 0.15 |
| hsa-let-7a | 0.16 | 0.07 | 0.98 | 0.02 | 0.16 |
| hsa-miR-134 | 0.21 | 0.06 | 1.25 | 0.20 | 0.17 |
| hsa-miR-486-5p | 0.91 | 0.06 | 4.37 | 0.05 | 0.21 |
| hsa-miR-21 | 0.45 | 0.04 | 0.09 | 0.03 | 4.77 |
| hsa-miR-205 | 7.82 | 0.09 | 1.77 | 0.05 | 4.41 |
| hsa-miR-218 | 0.12 | 0.09 | 0.5 | 0.20 | 0.25 |
| hsa-miR-194 | 0.93 | 0.86 | 0.23 | 0.02 | 4.04 |
| hsa-miR-151-3p | 0.83 | 0.23 | 0.26 | 0.05 | 3.19 |

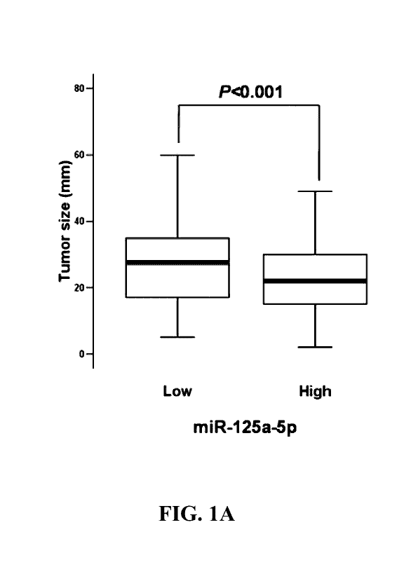

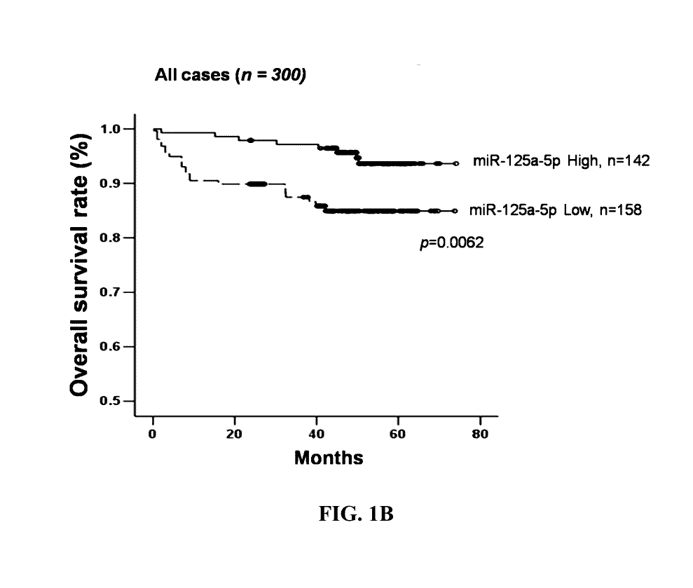

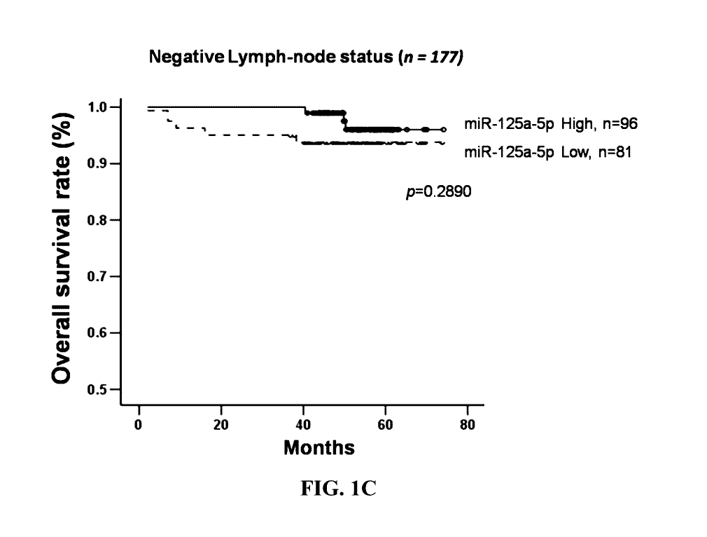

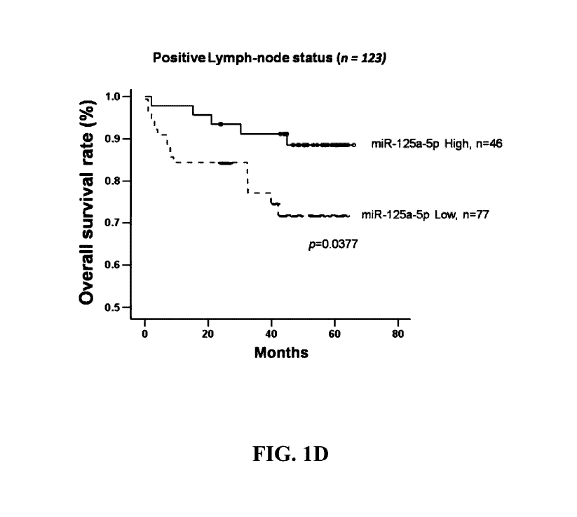

To further understand the significance of miR-125a-5p expression in breast cancer patients, serum levels of miR-125a-5p were measured in the sera of 300 breast cancer patients by quantitative RT-PCR (qRT-PCR). We used median Ct miRNA expression level to define the high and low categories. Patients were stratified into two groups based on the dichotomized scores (Table 2): high expression, miR-125a-5p expression >median (n=142 patients); low expression, miR-125a-5p expression <or =median (n=158 patients). The analysis showed that miR-125a-5p expression was inversely and significantly correlated with clinicopathological parameters including tumor grade, lymph-node status (Table 2), and tumor size (FIG. 1A). Low miR-125a-5p expression was associated with lower survival rates (FIG. 1B). Patients with positive lymph nodes (n=123 patients) had the worst survival rate (FIG. 1D) compared to patients with negative lymph nodes (n=177 patients, FIG. 1C).

TABLE 2

| | Number of | miR-125a-5p | | | |
|---|---|---|---|---|---|
| | cases | Low | High | | |
| Variables | (N = 300) | expression | expression | r-value | p-value |
| Stage | | | | −0.104 | 0.071 |
| I | 121 | 58 | 63 | | |
| II | 133 | 70 | 63 | | |
| III | 46 | 30 | 16 | | |
| Grade | | | | −0.167 | 0.004 |
| I | 128 | 54 | 74 | | |
| II | 106 | 64 | 42 | | |
| III | 66 | 40 | 26 | | |
| Lymph-node | | | | −0.166 | 0.004 |
| status | | | | | |
| Negative | 177 | 81 | 96 | | |
| Positive | 123 | 77 | 46 | | |
| Estrogen | | | | −0.045 | 0.436 |
| receptor status | | | | | |
| Negative | 101 | 50 | 51 | | |
| Positive | 199 | 108 | 91 | | |
| Progestone | | | | 0.016 | 0.785 |
| receptor status | | | | | |
| Negative | 125 | 67 | 58 | | |
| Positive | 175 | 91 | 84 | | |
| Her2/Neu status | | | | 0.108 | 0.061 |
| Negative | 202 | 114 | 88 | | |
| Positive | 98 | 44 | 54 | | |

Next, we performed multivariate Cox regression analysis with the clinicopathological parameters and miR-125a-5p expression. The level of miR-125a-5p expression was statistically significant predictors of breast cancer mortality.

TABLE 3

| | |
|---|---|
| Odds ratio | 95% CI |

| Variables | (OR) | Lower | Upper | p-value |
|---|---|---|---|---|
| miR-125a-5p | 0.421 | 0.184 | 0.961 | 0.040 |
| Stage | 2.405 | 1.324 | 4.368 | 0.004 |
| Grade | 1.131 | 0.565 | 2.261 | 0.729 |
| LN status | 1.249 | 1.026 | 1.520 | 0.027 |
| ER status | 0.909 | 0.698 | 1.184 | 0.479 |
| PR status | 0.870 | 0.664 | 1.139 | 0.310 |
| Her2/neu status | 1.004 | 0.866 | 1.259 | 0.648 |

miR-125a-5p Overexpression Decreases Cancer Cell Growth and Motility In Vitro

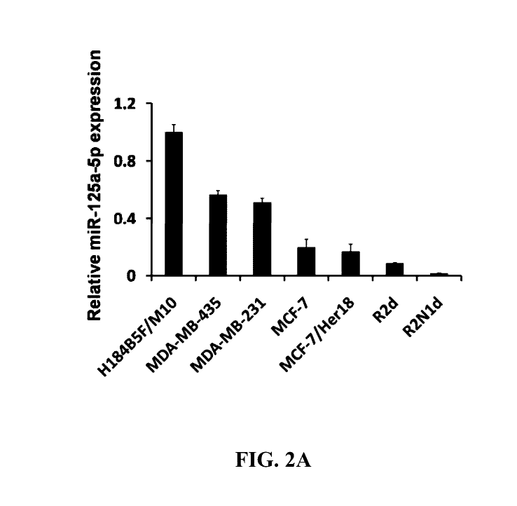

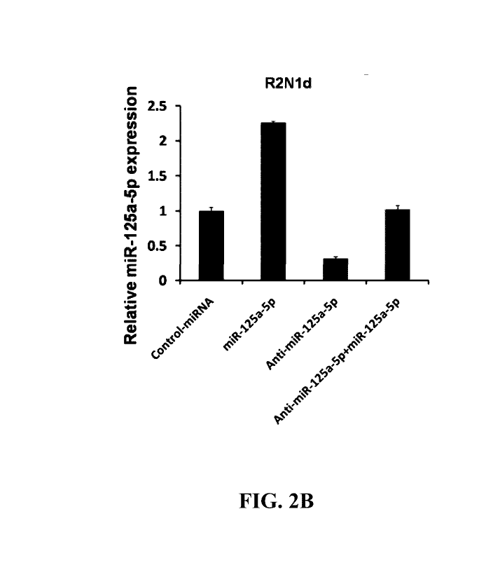

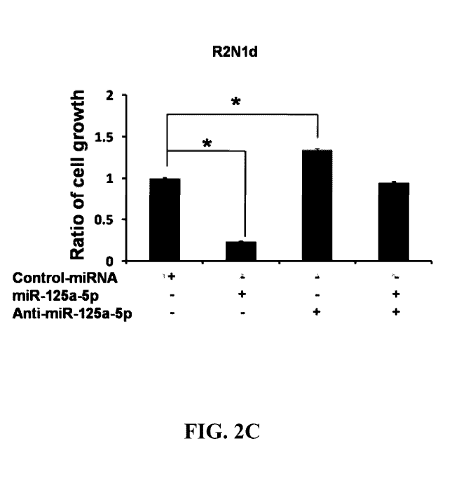

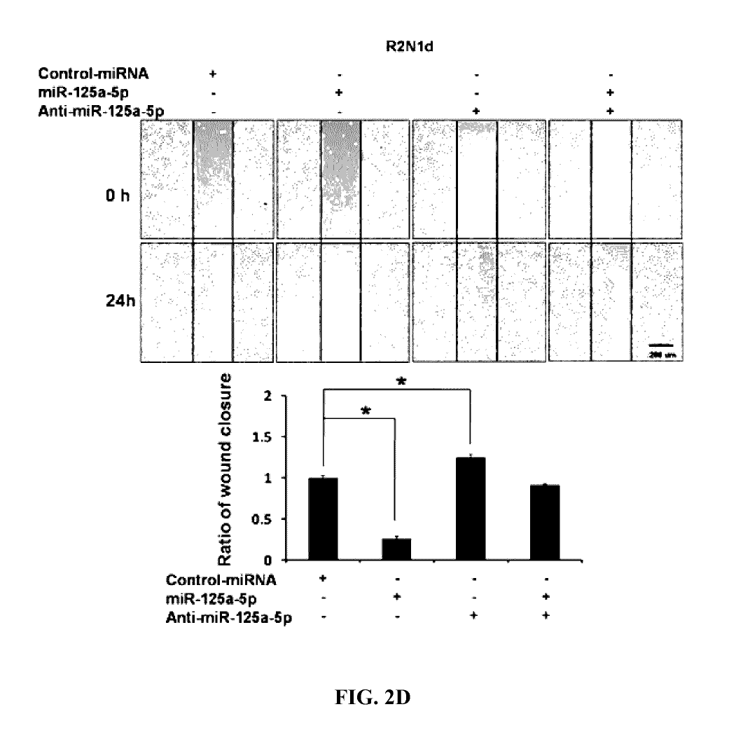

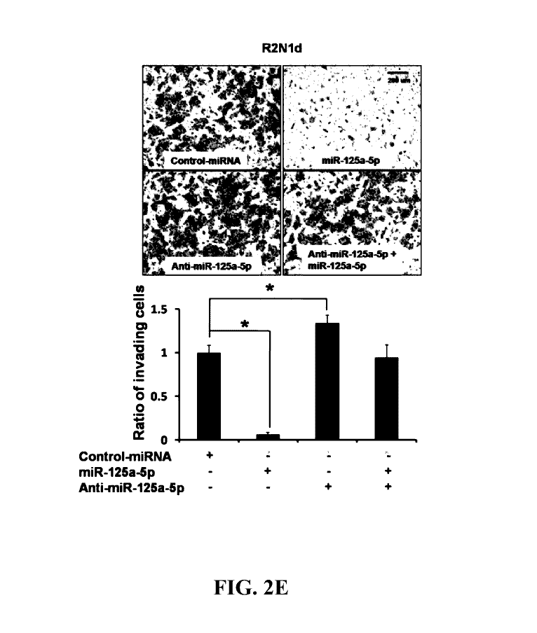

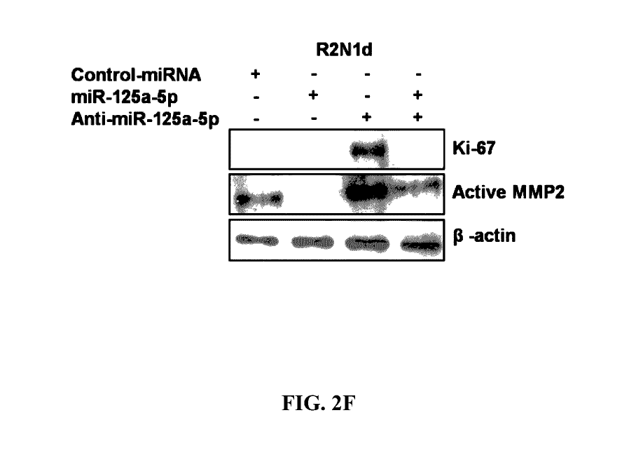

We first analyzed using qRT-PCR the expression of miR-125a-5p in different cell lines. Non-transformed breast epithelium cell line (H184B5F/M10) had the highest expression compared with malignant breast cancer cell lines (FIG. 2A). R2N1d, is a stem cell-like, highly malignant and metastatic cell line derived from human breast epithelial cells, had the lowest miR-125a-5p expression in the group.

To examine the cellular function of miR-125a-5p, we overexpressed or depleted miR-125a-5p in R2N1d (FIG. 2B). Down-regulation of miR-125a-5p promoted cells growth, migration, and invasion (FIGS. 2C, 2D, 2E). To further confirm the biological function of miR-125a-5p in cells growth and migration, the levels of Ki-67 and MMP2 was examined with Western analysis. Overexpression of miR-125a-5p decreased Ki-67 and active MMP2 levels in R2N1d cells (FIG. 2F).

These results together demonstrate an important role for miR-125a-5p in cells growth, migration and invasion of breast cancer cells.

HDAC4 is a Direct Target of miR-125a-5p

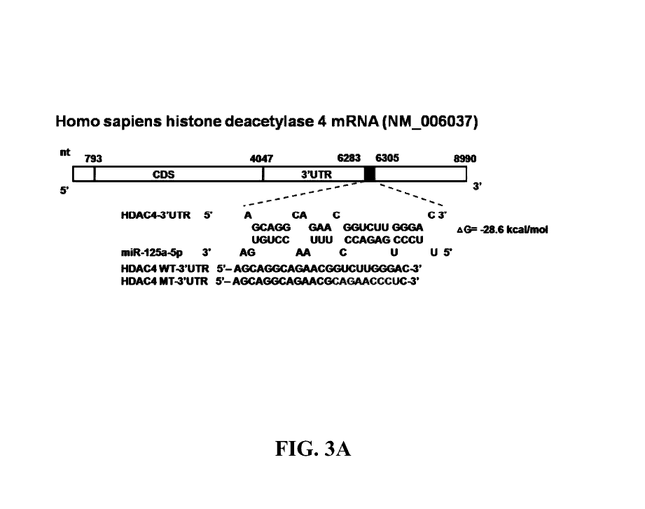

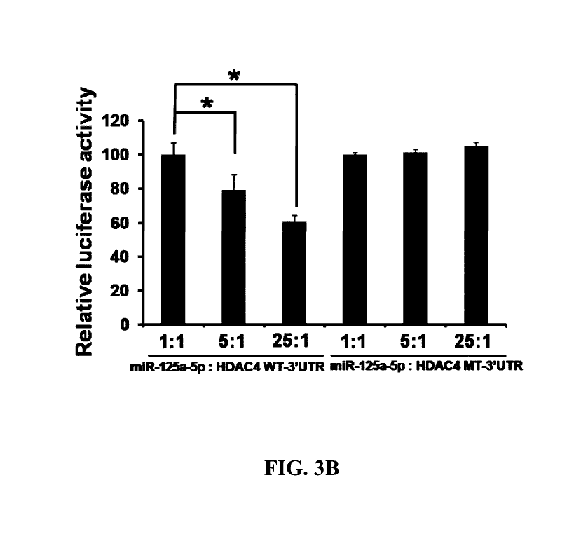

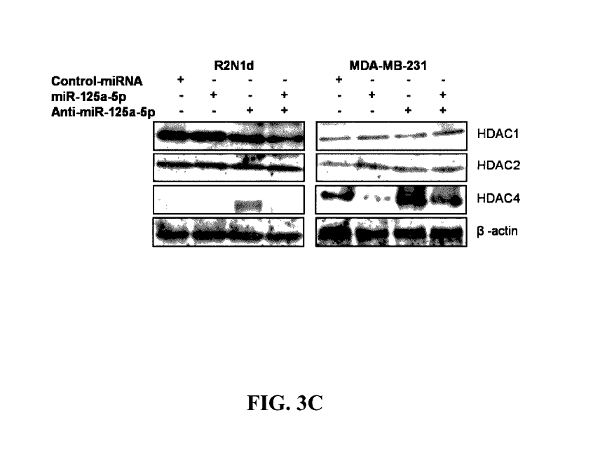

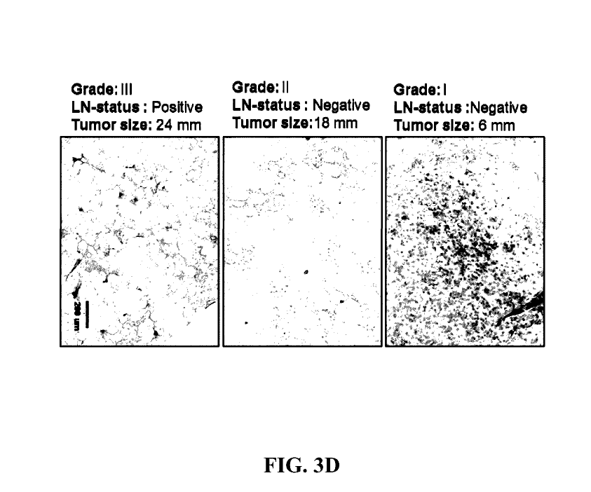

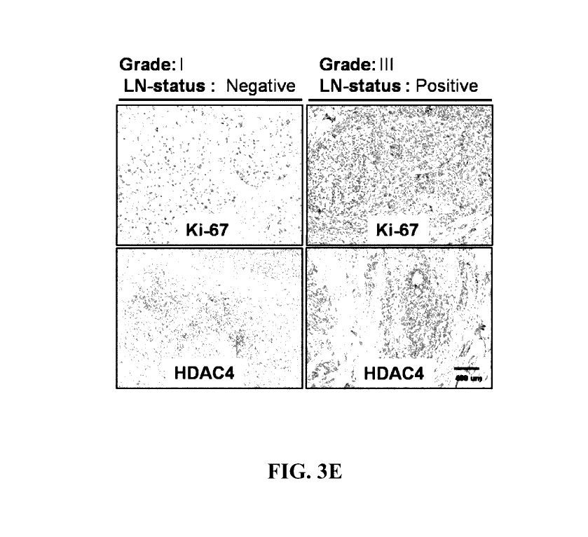

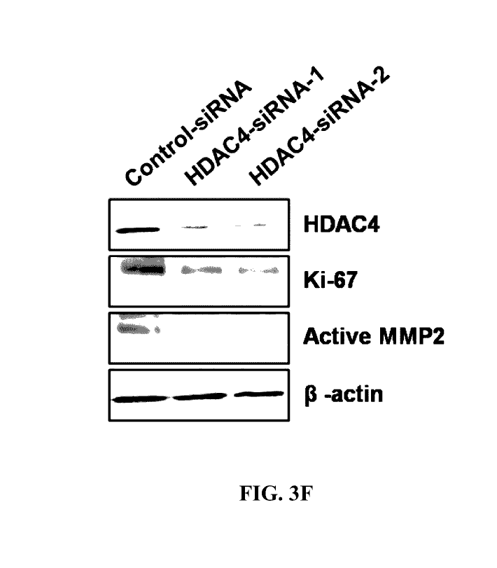

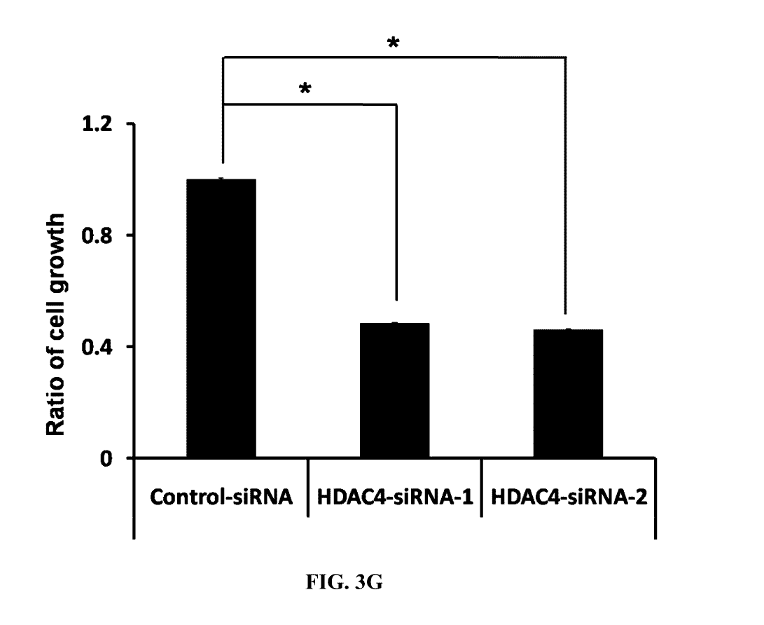

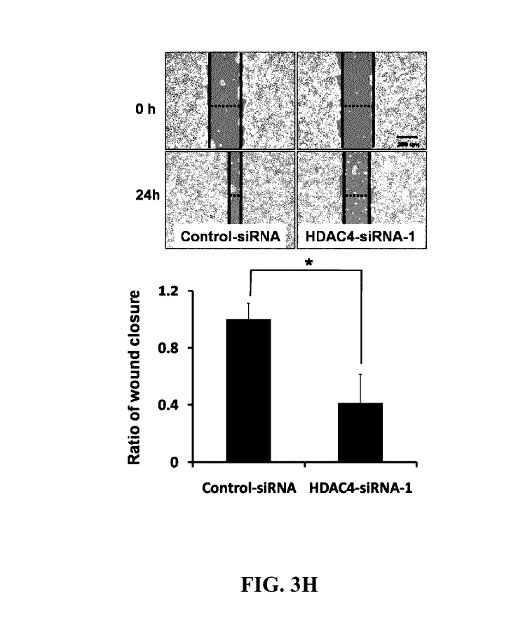

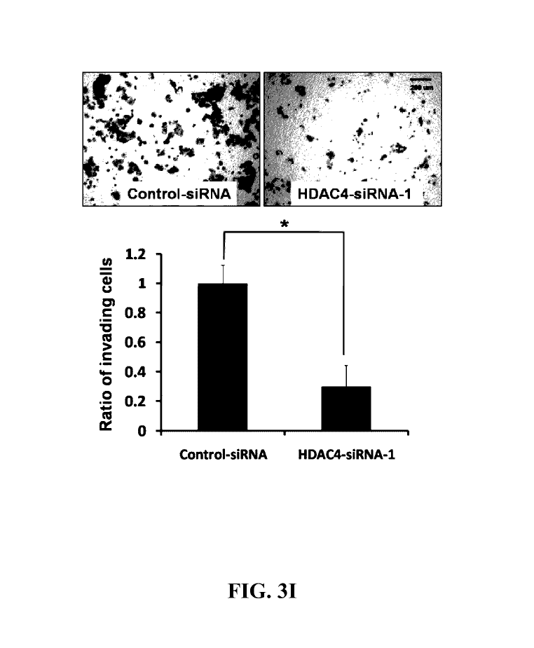

Calculation using TargetScan (Human 5.1) indicated the most thermodynamically favorable interactions between the 5′-end of miR-125a-5p and the 3′-UTR of the HDAC4 gene. We therefore hypothesized that miR-125a-5p may suppress HDAC4 expression by directly binding to the target sites within the 3′-UTR of the HDAC4 mRNA (FIG. 3A). To test this hypothesis, luciferase reporter vectors (PGL3) encoding wild-type (WT) and mutated (MT) 3′-UTRs of HDAC4 was constructed and co-transfected with a miR-125a-5p plasmid into HEK-293T cells. We found that miR-125a-5p suppressed the luciferase reporter activity in a dose-dependent manner (FIG. 3B). In contrast, the mutant HDAC4 construct, in which the miR-125a-5p target sequence was mutated, was unresponsive to miR-125a-5p.

This result was confirmed by Western analysis showing that miR-125a-5p overexpression decreased HDAC4 protein levels in vitro, but not HDAC1 or HDAC2, which do not contain the targeting sequence of miR-125a-5p in their mRNA sequences (FIG. 3C). These data indicate that miR-125a-5p directly targets HDAC4 in human breast cancer.

To examine the relationship between miR-125a-5p and HDAC4 in patients, in situ hybridization analysis was performed. The results showed that miR-125a-5p expression was highest in Grade I compared with Grade II and Grade III tissues (FIG. 3D). In contrast, HDAC4 expression was lowest in Grade I compared with Grade III tissues (FIG. 3E). Thus, miR-125a-5p is inversely correlated with HDAC4 in human breast tumors.

HDAC4 plays an important role in breast cancer growth and invasion. Depleting HDAC4 by RNA interference down-regulated the levels of Ki-67 and active MMP2 (FIG. 3F). Depleting HDAC4 also decreased cells growth, migration, and invasion in R2N1d (FIGS. 3G-3I) cells.

Overall, these results suggest that miR-125a-5p blocks tumor development by targeting HDAC4.

miR-125a-5p Decreases Growth, Metastasis, and Angiogenesis In Vivo

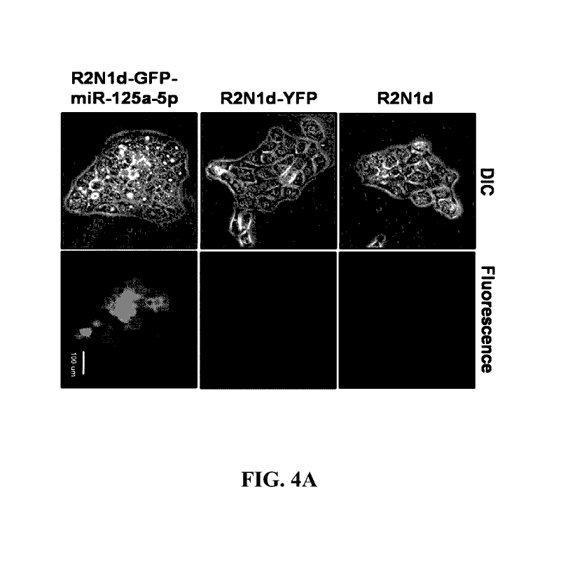

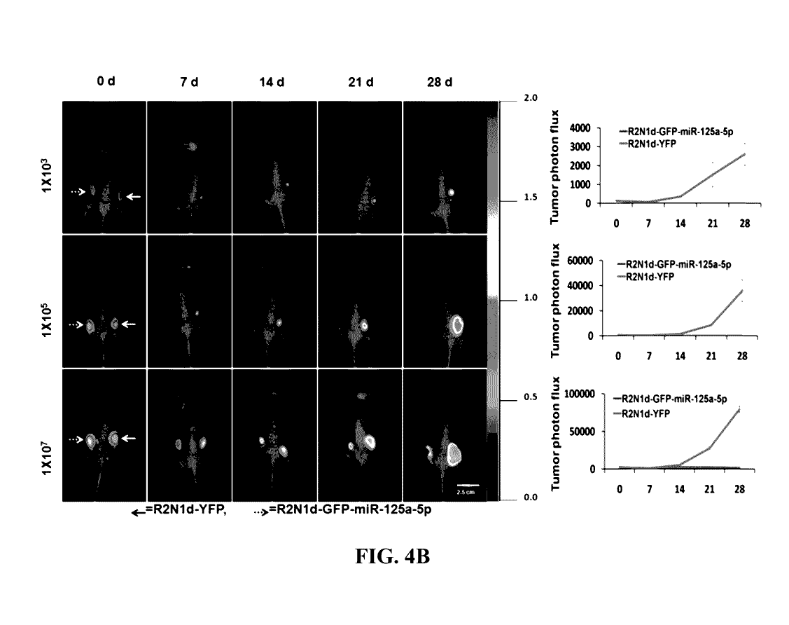

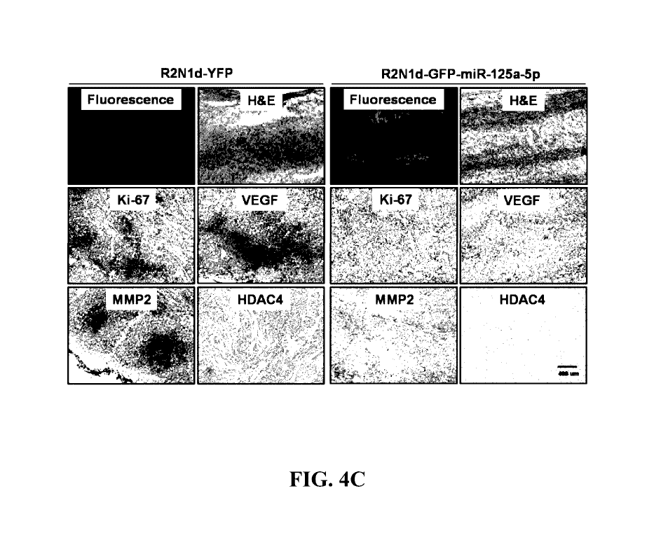

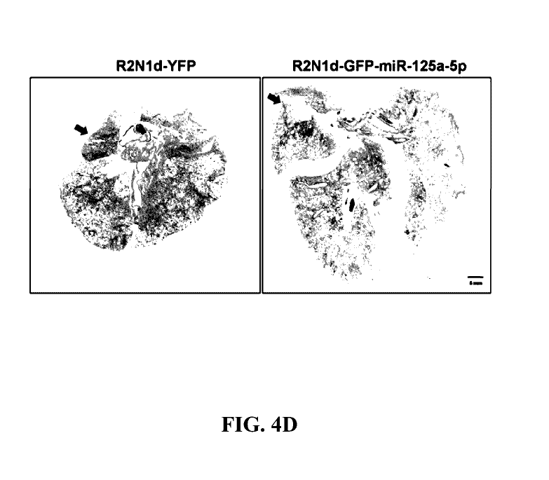

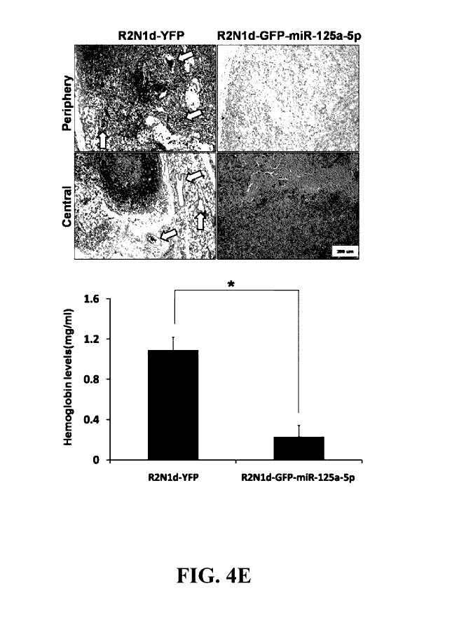

To test the tumor suppression function of miR-125a-5p, R2N1d cells were infected with lentivirus-encoded pLKO.1-YFP or pLKO.1-GFP-miR-125a-5p plasmid and stable clones were generated by puromycin selection. The R2N1d-GFP-miR-125a-5p cells, but not the R2N1d-YFP cells, exhibited membrane blebbing, a hallmark characteristic of apoptosis (FIG. 4A).

Tumorigenesis was tested by subcutaneous inoculation of different numbers (1×10³, 1×10⁵ or 1×10⁷) of R2N1d-YFP and R2N1d-GFP-miR-125a-5p cells into nude mice. Whole-body bioluminescence detection was used to detect tumor growth on day 0, 7, 14, 21, and 28 after inoculation. R2N1d-GFP-miR-125a-5p cells yielded a significant lower mean fluorescence intensity compared with the R2N1d-YFP cells (FIG. 4B). IHC staining revealed that expression of Ki-67, vascular endothelial growth factor (VEGF), and MMP2 was low in R2N1d-GFP-miR-125a-5p tumors compared to R2N1d-YFP tumors (FIG. 4C). Consistent with the finding that miR-125a-5p suppresses HDAC4 expression in vitro (FIG. 3), HDAC4 expression was higher in R2N1d-YFP tumors (FIG. 4C).

We then evaluated the role of miR-125a-5p during metastasis using a lung metastasis animal model in which R2N1d-YFP or R2N1d-GFP-miR-125a-5p cells were directly injected into the left lung through thorax of nude mice. One week after injection, the lungs were removed, and the metastatic nodules of the right lung were counted and the tissue sections were stained by hematoxylin and eosin (H&E). We found that the tumor metastasis into the intra- and extra-thoracic lymph nodes of right lung was not obvious (FIG. 4D).

TABLE 4

| | R2N1d-YEF | R2N1d-GFP-miR-125-5p |
|---|---|---|
| Extrathoracic lymph nodes | 14.4 ± 2.3 | 4 ± 1.0 |
| (Number of nodules in the right | | |
| lung) | | |
| Intrathoracic lymph nodes | 5/6 | 1/6 |
| (positively/mice number) | | |

To assess angiogenic potential of these cells, matrigel plug assay was performed in nude mice. R2N1d-GFP-miR-125a-5p cells produced fewer functional blood vessels and lower hemoglobin levels compared with R2N1d-YFP cells (FIG. 4E). These data demonstrate that miR-125a-5p blocks the ability of breast cancer cells to grow, metastasize, and develop blood vessels in vivo.

HDAC4 as a Therapeutic Target of miR-125a-5p

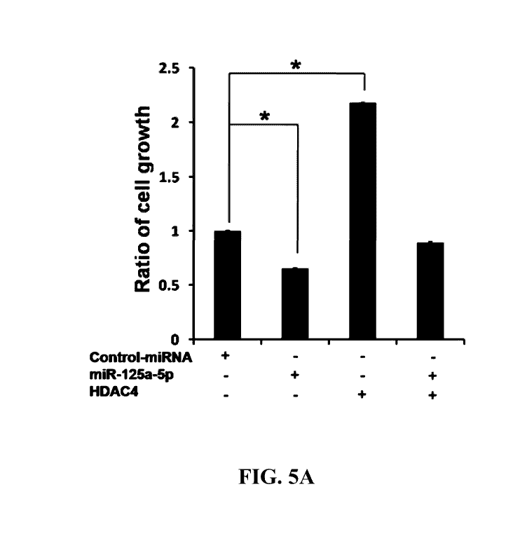

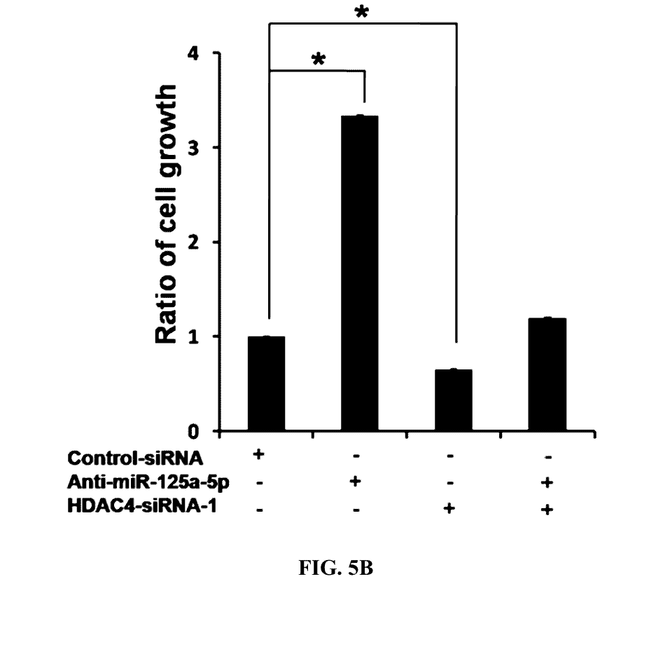

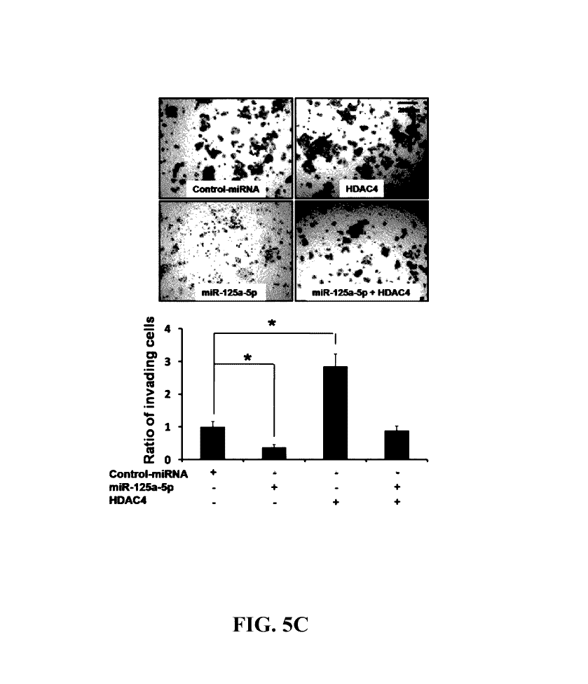

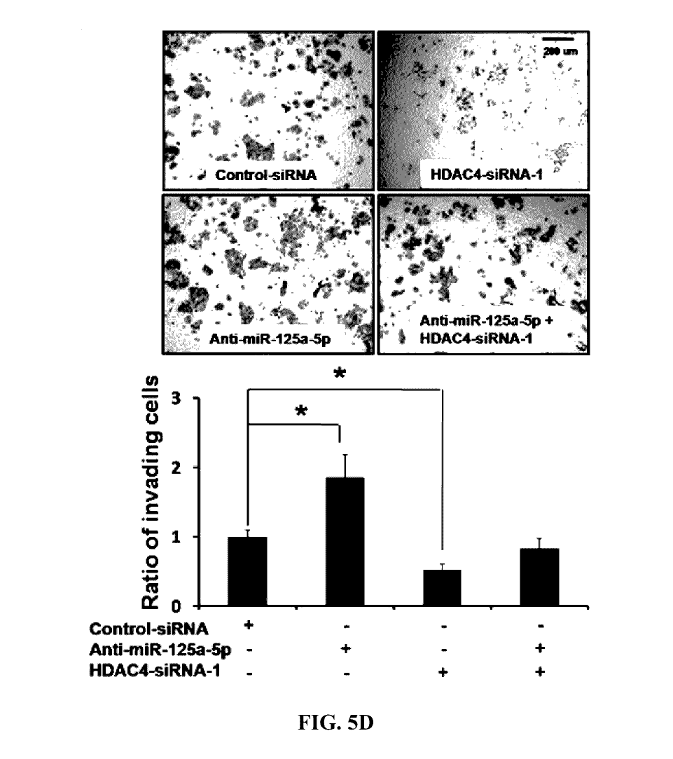

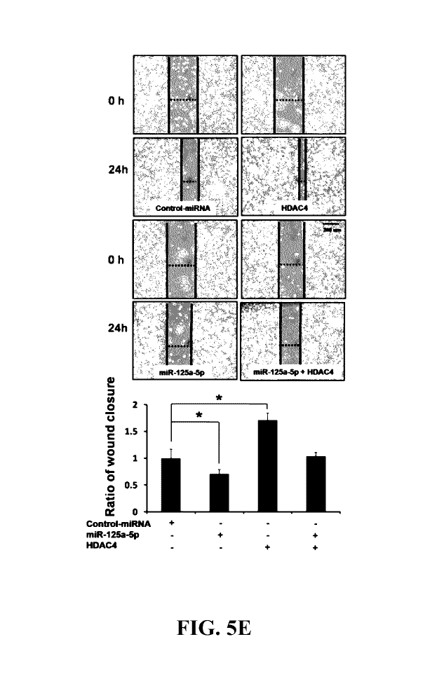

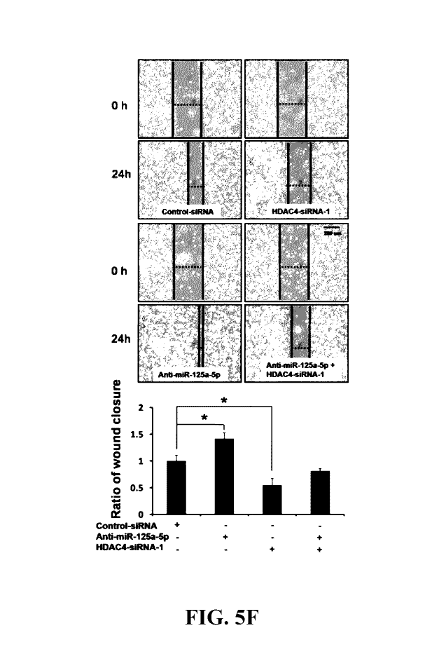

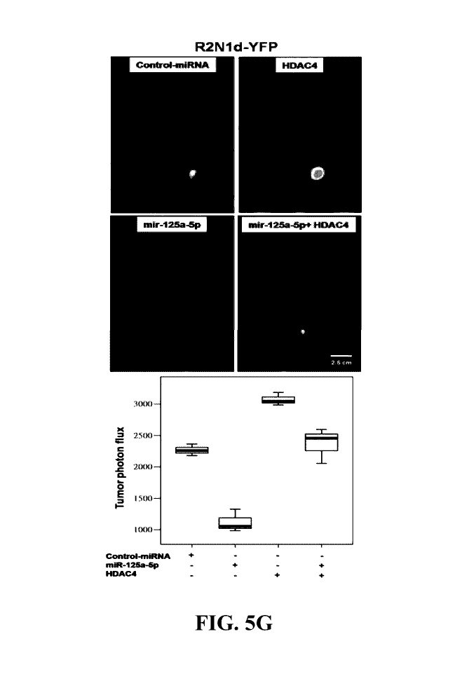

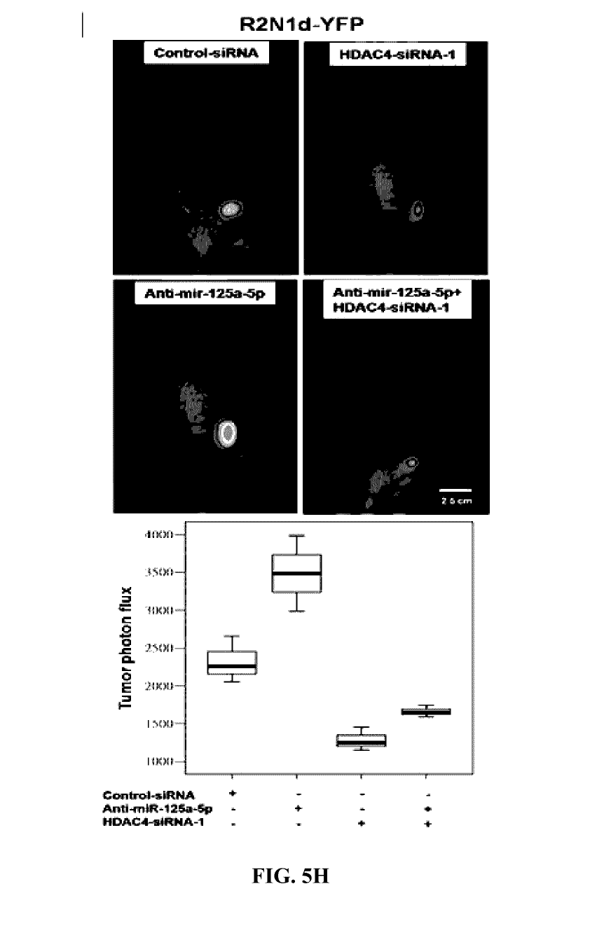

These results together suggest a counteracting mechanism of miR-125a-5p and HDAC4 in tumor development. Overexpression of HDAC4 abolished miR-125a-5p-mediated inhibition in growth (FIG. 5A), invasion (FIG. 5C), migration (FIG. 5E), tumor growth (FIG. 5G) and metastasis (FIG. 5I) in R2N1d cells. Conversely, silencing of HDAC4 abolish anti-miR-125a-5p-induced growth (FIG. 5B), invasion (FIG. 5D) and migration (FIG. 5F) as well as tumor growth/metastasis (FIGS. 5H, 5I). Importantly, HDAC4 is a functional target of miR-125a-5p to suppress growth and tumor progression of breast cancer.

TABLE 5

| | Control- | miR- | | miR-125a-5p + |
|---|---|---|---|---|
| | miRNA | 125-5p | HDAC4 | HDAC4 |
| Extrathoracic lymph | 12.2 ± 3.6 | 3.4 ± 1.1 | 22.6 ± 3.2 | 11.8 ± 2.4 |
| nodes (Number of | | | | |
| nodules in the | | | | |
| right lung) | | | | |
| Intrathoracic lymph | 4/6 | 0/6 | 5/6 | 3/6 |
| nodes (positively/ | | | | |
| mice number) | | | | |

TABLE 6

| | | | | Anti- |
|---|---|---|---|---|
| | | | | miR-125a- |
| | Control- | Anti-miR- | HDAC4- | 5p + HDAC4- |
| | miRNA | 125-5p | siRNA-1 | siRNA-1 |
| Extrathoracic lymph | 12 ± 1.6 | 25 ± 3.2 | 6.2 ± 0.8 | 14.4 ± 1.5 |
| nodes (Number of | | | | |
| nodules in the | | | | |
| right lung) | | | | |
| Intrathoracic lymph | 5/6 | 6/6 | 2/6 | 4/6 |
| nodes (positively/ | | | | |
| mice number) | | | | |

While the invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A method for inhibiting activity of histone deacetylase 4 (HDAC4), comprising:
contacting a miR-125a-5p with a cell in need thereof.

2. The method of claim 1, wherein the cell is a breast cancer cell.

3. The method of claim 2, wherein the breast cancer cell is an H184B5F/M10 cell, an MDA-MB-435 cell, an MDA-MB-231 cell, an MCF-7 cell, or an MCF-7/Her18 cell.

4. A method for treating a breast cancer, comprising:
administering a miR-125a-5p to a breast cancer patient.

5. The method of claim 4, wherein the miR-125a-5p suppresses growth, invasion, migration, metastasis, and/or angiogenesis of a breast cancer cell.

6. The method of claim 4, wherein the miR-125a-5p suppresses expression level of HDAC4 in a breast cancer cell.
